(19) United States
(12) Patent Application Publication
(10) Pub. No.: US 2013/0158454 A1
(43) Pub. Date: Jun. 20, 2013

(54) Apparatus and method for controlling pain

(71) Applicant: Jeffrey McFarlane, Vero Beach, FL (US)

(72) Inventor: Jeffrey McFarlane, Vero Beach, FL (US)

(21) Appl. No.: 13/374,169

(22) Filed: Dec. 14, 2011

(51) Int. Cl. A61H 1/00; A61B 17/122

(52) U.S. Cl. 601/70; 606/201

(57) ABSTRACT

A pain controlling device includes a pair of housings, each having a jaw at one end and a handle at the other end. The jaws, which are held together a resilient coupling connecting the housings, may be separated by squeezing the handles together. Each of the housings includes a source of mechanical vibration that can be turned on to control pain in an extremity that is clamped between the jaws, such as in a finger from which a blood sample is to be taken.

### RELATED APPLICATIONS

Not Applicable.

### FEDERALLY SPONSORED RESEARCH OR DEVELOPMENT

Not Applicable.

### BACKGROUND OF THE INVENTION

1. Field of the Invention

This invention relates to an apparatus and method for controlling pain by applying mechanical vibrations to parts of the body.

2. Summary of the Background Information

The patent literature includes a number of descriptions of devices that apply vibration to specific areas of the body to achieve a degree of pain management or control. Such devices appear to work by tending to overwhelm the sensory system, reducing the ability of the pain messages to pass through the system. For example a source of vibration may be placed in a box structure strapped to the wrist of a user like a wrist watch, or a source of vibration may be placed in a box having a plurality of flexible arms with enlarged spherical ends to be held against the user's body.

### SUMMARY OF THE INVENTION

According to one aspect of the invention, a device for controlling pain is provided, including a pair of housings, a vibration source, a resilient coupling, and control means. The housings each include a jaw at a distal end, a handle at a proximal end, and a vibration source. The resilient coupling, which attaches the housings to one another between the handle and the jaw of each of the housings, holds the jaws of the housings close to one another, while squeezing the handles together moves the jaws of the housings away from one another.

Preferably, the apparatus for controlling pain additionally includes at least one battery producing electrical power, while the vibration source within each of the housings includes an electric motor and an eccentric weight rotationally driven by the motor, and while the control means includes at least one switch connecting the electric motor within each of the housings to electrical power. Each of the housings may include a battery and a switch connecting the battery within the housing to the electric motor within the housing. Each of the housings may additionally include an indicator light connected to the battery within the housing through the switch within the housing.

In a first embodiment, the resilient coupling includes a U-shaped coupling bracket and a pair of cantilever springs. The U-shaped coupling bracket has a cross member and a pair of legs, each of which extends from an end of the cross member along an adjacent housing within the pair of housings toward the distal end of the adjacent housing. Each of the cantilever springs is attached to the coupling bracket to extend along the adjacent housing toward the distal end of the adjacent housing. A distal end of each of the cantilever springs is attached to the adjacent housing to hold the jaws of the housings close to one another. Each of the housings is pivoted in contact with the coupling bracket to move the jaw of the housing away from the jaw of the other housing. Preferably, each of the cantilever springs includes a concave surface facing the other cantilever spring, with the concave surfaces of the cantilever springs facing one another to form a rounded opening between the cantilever springs. The cantilever springs may include additional concave surfaces, facing one another to form additional rounded openings between the cantilever springs.

In a second embodiment, the jaw of each of the housings includes a concave surface facing the jaw of the other housing, with the concave surfaces of the jaws facing one another to form a rounded opening between the jaws. The jaw of each of the housings may include a plurality of concave surfaces extending along the jaw, facing one another to from a plurality of rounded openings between the jaws. The resilient coupling may include a resilient bracket attached to each of the housings and extending between the housings. Alternately, the resilient coupling includes a U-shaped coupling bracket having a cross member and a pair of legs, wherein each of the legs extends from an end of the cross member along an adjacent housing within the pair of housings; and a resilient block held between each of the housings and an adjacent leg of the coupling bracket.

According to another aspect of the invention, a device for controlling pain includes first and second concave surfaces, a connecting structure, a pair of handles. a first vibration source, and a first control switch. The connecting structure, which connects the first and second concave surfaces to face one another, includes a resilient coupling holding the concave surfaces close to one another, while squeezing the handles together moves the concave surfaces apart from one another. The first control switch controls the first vibration source to operate, causing the first concave surface to vibrate.

According to yet another aspect of the invention, a method for controlling pain within a body part includes steps of: squeezing handles within a pain controlling device together to move concave surfaces within the pain controlling device apart from one another; moving the concave surfaces within the pain controlling device into place around opposite sides of the body part, releasing the handles to allow a resilient member within the pain controlling device to move the concave surfaces toward one another over the body part; and switching on a first vibration source within the pain controlling device to cause vibration within a first of the concave surfaces.

The method may additionally include switching on a second vibration source within the pain controlling device. The method may further include puncturing a portion of the skin of the body part after switching on the first vibration source.

### BRIEF DESCRIPTION OF THE DRAWINGS

These and other aspects of the invention will be made apparent by reading the following specification in conjunction with the accompanying figures, in which:

FIG. 1 is a partly sectional plan view of a pain controlling device built in accordance with a first embodiment of the invention;

FIG. 2 is a plan view of the pain controlling device of FIG. 1, shown with upper covers of housings therein removed;

FIG. 3 is a plan view of a pain controlling device built in accordance with a second embodiment of the invention;

FIG. 4 is a cross-sectional plan view of an alternative resilient coupling for use in the pain controlling device of FIG. 3;

FIG. 5 is a schematic view of a first alternative electrical circuit for use in the pain controlling devices of FIGS. 1 and 3;

FIG. 6 is a schematic view of a second alternative electrical circuit for use in the pain controlling devices of FIGS. 1 and 3; and

FIG. 7 is a fragmentary perspective view showing a use of the pain controlling device of FIG. 3.

### DETAILED DESCRIPTION OF THE INVENTION

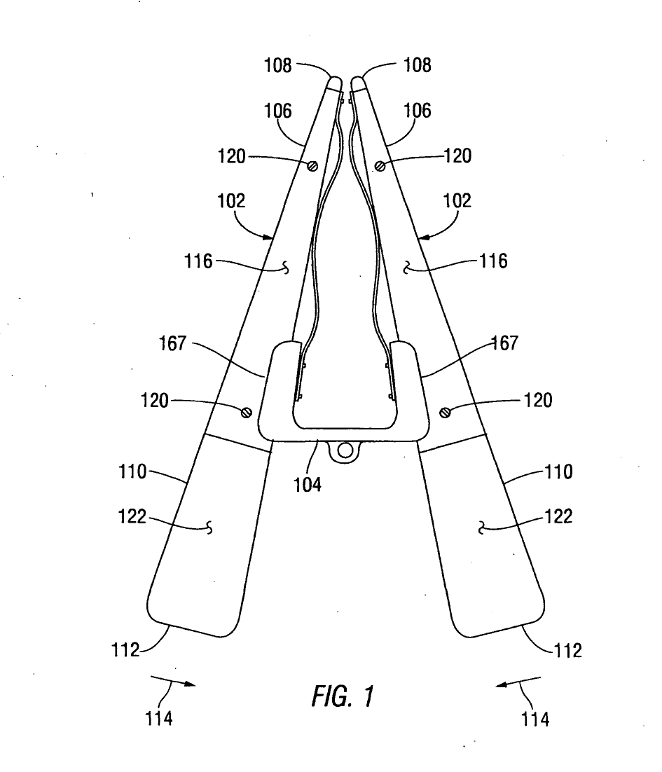

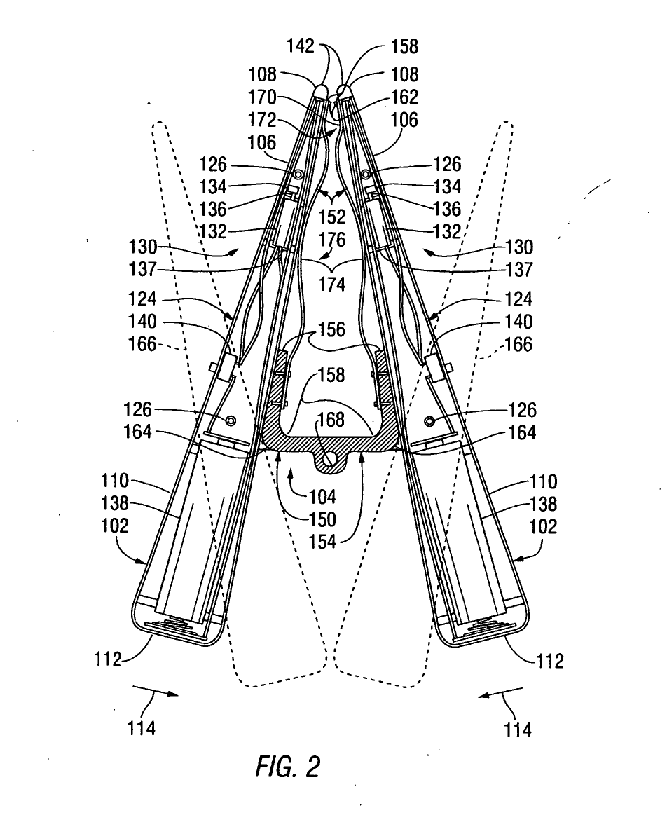

FIG. 1 is a partly sectional plan view of a pain controlling device 100, built in accordance with a first embodiment of the invention to include a pair of housings 102 and a resilient coupling 104. Each of the housings 102 includes a jaw 106 extending to a distal end 108 and a handle 110 extending to a proximal end 112. The resilient coupling 104, which attaches the housings 102 to one another between the jaw 106 and the handle 110 of each housing 102, holds the jaws 106 of the housings 102 close to one another, while squeezing the handles 110 together, in the directions of arrows 114, moves the jaws 106 of the housings 102 away from one another. Each of the housings 102 includes an upper cover 116, attached to a lower cover 118 (shown in FIG. 2) by screws 120, and a battery cover 122, which is snapped into place.

FIG. 2 is a plan view of the pain controlling device 100, shown with the upper cover 116 and the battery cover 122 (both shown in FIG. 1) of each housing 102 removed to reveal elements held within a lower cover 124 of the housing 102. The lower cover 124 includes holes 126 provided to engage the screws 120 to fasten the upper cover 116 in place. Each housing 102 includes a vibration source 130 formed by an electric motor 132 driving an eccentric weight 134 mounted on a drive shaft 136 in rotation. The electric motor 102 is held within ribs 137 of the lower cover 124, with these ribs 137 transferring vibration of the motor 102, resulting from the rotation of the eccentric weight 134 to the housing 102. Each housing 102 additionally includes a battery 138, producing electrical power, connected to the electric motor 132 through a switch 140, which serves as a control means turning the electric motor 132 on and off. In the example of the figures, each housing 102 further includes an indicator light 142, such as a light emitting diode (LED), additionally connected to the battery 138 through the switch 140, producing a visible indication whenever the electric motor 132 is turned on.

The resilient coupling 104 includes a U-shaped coupling bracket 150 and a pair of cantilever springs 152. The U-shaped coupling bracket 150 has a cross member 154 and a pair of legs 156, each of which extends from an end 158 of the cross member 154 along a housing 102 adjacent to the leg 156, toward the distal end 108 of the adjacent housing 102. The cross member 154 includes a hole 158 Each of the cantilever springs 152 is attached to the coupling bracket 150 to extend along the adjacent housing 102 toward the distal end 108 of the adjacent housing 102. A distal end 158 of each of the cantilever springs 152 is attached to the adjacent housing 102 by screws 162 to hold the jaws 106 of the housings 102 close to one another. Each of the housings 102 is pivoted in contact with a surface 164 of the coupling bracket 150 into a position indicated by dashed lines 166, moving the jaw 106 of the housing 102 away from the jaw 106 of the other housing 102. The U-shaped coupling bracket 150 may additionally include flanges 167, shown in FIG. 1, to control the lateral alignment of the housings 102 on the U-shaped coupling bracket 150, which preferably additionally includes a hole 168, which is provided for attachment to a belt clip or a lanyard.

Each of the cantilever springs 152 includes a concave surface 170 facing the other cantilever spring 152, with the concave surfaces 170 of the cantilever springs 152 facing one another to form a rounded opening 172 between the cantilever springs 152. The cantilever springs include additional concave surfaces 174, facing one another to form an additional rounded opening 176 between the cantilever springs 152. While the smaller rounded opening 172 is configured to fit around a small body part, such as a finger, the larger rounded opening 176 is configured to fit around a larger body part, with vibrations transmitted from the concave surfaces 206, 210 controlling pain within the body part.

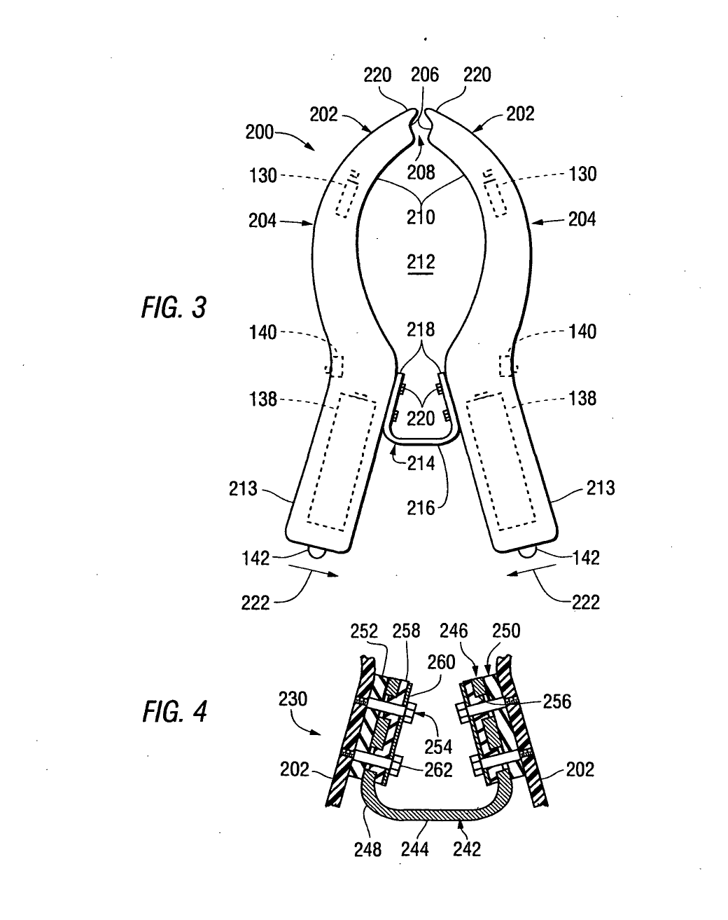

FIG. 3 is a plan view of a pain controlling device 200 built in accordance with a second embodiment of the invention to include a pair of housings 202, each including a jaw 204 having a concave surface 206 facing the jaw 204 of the other housing 202, with the concave surfaces 206 facing one another to form a rounded opening 208 between the jaws 204. In the example of the figure, the jaw 204 of each of the housings 202 additionally includes a second concave surface 210, with the concave surfaces 210 facing one another to form a second rounded opening 212 between the jaws 204. The concave surfaces 206 are configured to fit around a small body part, such as a finger, while the larger concave surfaces 210 are configured to fit around a larger body part, such as a wrist, with vibrations transmitted from the concave surfaces 206, 210 controlling pain within the body part.

The pain controlling device 200 additionally includes a resilient coupling 214, in the form of a resilient bracket 216 including legs 218 attached to each of the housings 202 by screws 220 and a cross member 222 extending between the legs 218. Each of the housings 202 also includes a handle 213 and elements arranged to operate as described in detail above in reference to FIG. 2, which are therefore accorded line reference numbers, with such elements including a vibration source 130, a battery 140, a switch 48, and an indicator light 142. While the resilient bracket 216 holds the distal ends 220 of the jaws 204 close to one another, these distal ends 220 are moved apart as the handles 213 are squeezed together in the directions of arrows 222. This arrangement has an advantage of maintaining a space between the handles 213 that is sufficient to prevent pinching a user squeezing the handles 213 together.

FIG. 4 is a cross-sectional plan view of an alternative resilient coupling 230 for use in the pain controlling device 200. The alternative resilient coupling 230 includes a U-shaped coupling bracket 242 having a cross member 244 and a pair of legs 246, with each of the legs 246 extending from an end 248 of the cross member 244 along an adjacent housing 202 within the pair of housings 202. The alternative resilient coupling 230 additionally includes a pair of vibration isolation structures 250, each including an inner resilient block 252 held between each of the housings 202 and an adjacent leg 246 of the coupling bracket 242 by a pair of screws 254 extending through holes 256 in the adjacent leg 246. Preferably, each of the vibration isolation structures 250 further includes an outer resilient block 258 held in place against the leg 246 by a plate 260 extending under the heads 262 of the screws 254. For example, the resilient blocks 252 are provided to absorb vibrations, allowing the housings 202 to vibrate independently, while the coupling bracket 242 deflects when the handles 213 are squeezed together to move the distal ends 220 of the of the jaws 204 apart.

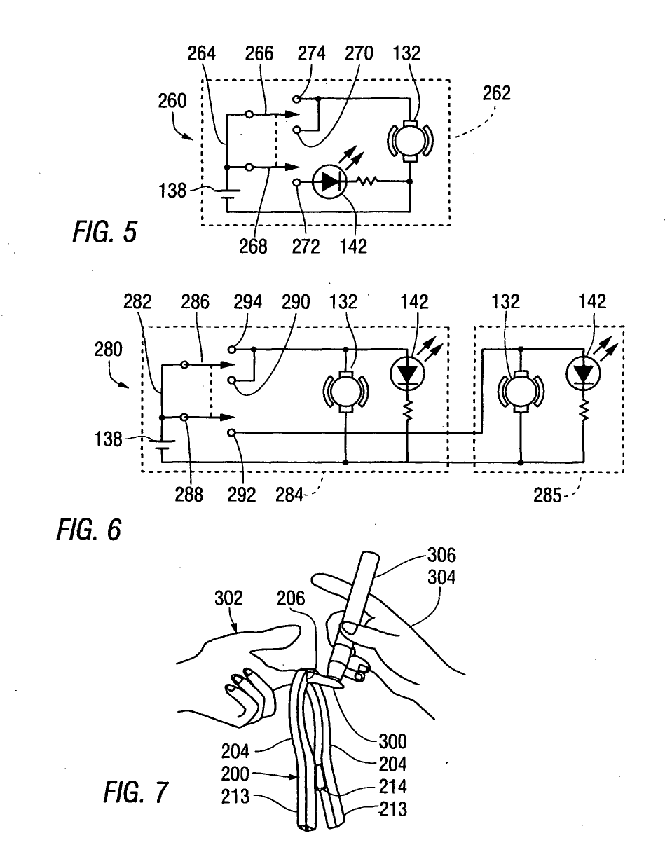

FIG. 5 is a schematic view of a first alternative electrical circuit 260 for a housing 262, which is otherwise as described above as housing 102 or housing 202. While the housings 102, 202 have previously been described as having a two-position switch 140 to turn the electric motor 132 and the indicator light 142 on and off simultaneously, the first alternative electrical circuit 260 includes a three position switch 264 providing a means for controlling whether the indicator light 142 is turned on with the electric motor 132. Specifically, if the center-off contacts 266, 268 are moved together into contact with contacts 270, 272, respectively, both the electric motor 132 and the indicator light 142 are turned on. If the center-off contact 266 is moved into contact with contact 272, only the electric motor 132 is turned on.

FIG. 6 is a schematic view of a second alternative electrical circuit 280, in which a single three-position switch 282 is used to control electrical motors 132 and indicator lights 142 in each of two housings 284, 285. If the center-off contacts 286, 288 are moved together into contact with contacts 290, 292, respectively, both the electric motor 132 and the indicator light 142 in both the housings 284, 285 are turned on. If the center-off contact 286 is moved into contact with contact 292, only the electric motor 132 and the indicator light 142 within the housing 284 is turned on.

FIG. 7 is a fragmentary perspective view showing an exemplary use of the pain controlling device 200 to control the pain of a puncturing the skin to obtain a blood sample. Persons with diabetes need to perform this process one or more times every day to ensure that blood sugar is being controlled properly. To use the method of the invention, the user first installs the pain controlling device 200 on the finger 300 of a hand 302 that will be used to obtain the blood sample by moving the device 200 into place with the handles 213 squeezed together, so that the concave surfaces 206 of the housings 204 are on opposite sides of the finger 300. Then the handles 213 are released so that the resilient coupling 214 moves the concave surfaces 206 into contact with opposite sides of the finger 300, holding the pain controlling device 200 in place, and allowing the other hand 304 can be used to perform the skin puncture using a conventional lancing device 306. After this process is complete, the handles 213 are again squeezed together to remove the pain controlling device 200 from the finger 300. While the pain controlling device 200 is shown as being applied to a human, it is understood that it can additionally be applied to animals.

It is understood that, while various parts and features have been described with some degree of particularity, such descriptions have been given only by way of example, and that many variations are possible within the spirit and scope of the inventions, which is understood to be limited only by the appended claims.

What is claimed is:

1. An apparatus comprising:
a pair of housings, each including a jaw at a distal end, a handle at a proximal end, and a vibration source;
a resilient coupling attaching the housings to one another between the handle and the jaw of each of the housings, wherein the resilient coupling holds the jaws of the housings close to one another, and wherein squeezing the handles together moves the jaws of the housings away from one another, and
control means controlling operation of the vibration source held within each of the housings.

2. The apparatus of claim 1, wherein the resilient coupling comprises:
a U-shaped coupling bracket having a cross member and a pair of legs, wherein each of the legs extends from an end of the cross member along an adjacent housing within the pair of housings toward the distal end of the adjacent housing, and
a pair of cantilever springs, each attached to the coupling bracket to extend along the adjacent housing toward the distal end of the adjacent housing, wherein a distal end of each of the cantilever springs is attached to the adjacent housing to hold the jaws of the housings close to one another, and wherein each of the housings is pivoted in contact with the coupling bracket to move the jaw of the housing away from the jaw of the other housing.

3. The apparatus of claim 2, wherein
each of the cantilever springs includes a concave surface facing the other cantilever spring, and
the concave surfaces of the cantilever springs face one another to form a rounded opening between the cantilever springs.

4. The apparatus of claim 2, wherein each of the cantilever springs includes a plurality of concave surfaces extending along the cantilever spring, and
the plurality of concave surfaces of the cantilever springs face one another to form rounded openings between the cantilever springs.

5. The apparatus of claim 1, wherein the resilient coupling includes a resilient bracket attached to each of the housings and extending between the housings.

6. The apparatus of claim 1, wherein the resilient coupling includes:
a U-shaped coupling bracket having a cross member and a pair of legs, wherein each of the legs extends from an end of the cross member along an adjacent housing within the pair of housings; and
a resilient block held between each of the housings and an adjacent leg of the coupling bracket.

7. The apparatus of claim 1, wherein
the jaw of each of the housings includes a concave surface facing the jaw of the other housing, and
the concave surfaces of the jaws face one another to form rounded openings between the jaws.

8. The apparatus of claim 2, wherein the jaw of each of the housings includes a plurality of concave surfaces extending along the jaw, and
the plurality of concave surfaces of the jaws of the housings face one another to form rounded openings between the jaws.

9. The apparatus of claim 1, additionally comprising at least one battery producing electrical power, wherein
the vibration source within each of the housings includes a electric motor and an eccentric weight rotationally driven by the motor, and
the control means includes at least one switch connecting the electric motor within each of the housings to electrical power.

10. The apparatus of claim 9, wherein each of the housings includes a battery and a switch connecting the battery within the housing to the electric motor within the housing.

11. The apparatus of claim 10, wherein each of the housings additionally includes an indicator light connected to the battery within the housing through the switch within the housing.

12. The apparatus of claim 11, wherein the switch includes separate positions for operating the motor within the housing and the indicator light within the housing.

13. A method for controlling pain within a body part, comprising:
squeezing handles within a pain controlling device together to move concave surfaces within the pain controlling device apart from one another;
moving the concave surfaces within the pain controlling device into place around opposite sides of the body part;
releasing the handles to allow a resilient member within the pain controlling device to move the concave surfaces toward one another over the body part; and
switching on a first vibration source within the pain controlling device to cause vibration within a first of the concave surfaces.

14. The method of claim 13, additionally comprising switching on a second vibration source within the pain controlling device.

15. The method of claim 13, additionally comprising puncturing a portion of skin of the body part after switching on the first vibration source.

16. An apparatus comprising:
first and second concave surfaces;
a structure connecting the first and second concave surfaces to face one another, wherein the structure includes a resilient coupling holding the concave surfaces close to one another;
a pair of handles, wherein squeezing the handles together moves the concave surfaces apart from one another;
a first vibration source causing the first concave surface to vibrate, and
a first control switch controlling the first vibration source.

17. The apparatus of claim 16, additionally comprising:
a second vibration source causing the second concave surface to vibrate; and
a second vibration source causing the second concave surface to vibrate.

18. The apparatus of claim 17, additionally comprising:
a third concave surface, connected to move with the first concave surface;
a fourth concave surface, facing the second concave surface and connected to move with the second concave surface.
